(19) United States
(12) Patent Application Publication
(10) Pub. No.: US 2011/0076256 A1
(43) Pub. Date: Mar. 31, 2011

(54) Human Neural Stem Cell and Pharmaceutical Composition for the Treatment of Central or Peripheral Nervous System Disorders or Injuries Using Same

(75) Inventor: Kook In Park, Seoul (KR)

(73) Assignee: Industry-Academic Cooperation Foundation, Yonsei University, Seoul (KR)

(21) Appl. No.: 12/994,953

(22) PCT Filed: Aug. 12, 2009
(86) PCT No.: PCT/KR09/04504

§ 371 Date: Nov. 29, 2010

(30) Foreign Application Priority Data
Aug. 12, 2008 (KR) 10-2008-0078927

(51) Int. Cl. A61K 35/12; A61P 25/16; A61P 25/28; A61P 25/08; A61P 25/00

(52) U.S. Cl. 424/93.7

(57) ABSTRACT

The present invention relates to a human neural stern cell, and to a pharmaceutical composition for the treatment of central or peripheral nervous system disorders and injuries using same. More particularly, the present invention relates to a human telencephalon-derived human neural stem cell effective in the treatment of nervous system disorders and injuries, and to a pharmaceutical composition for the treatment of nervous system disorders and injuries using same, to the use of the human neural stem cell for preparing therapeutic agents for the treatment of nervous system disorders and injuries, and to a method for treating nervous system disorders and injuries, capable of administrating an effective amount of the human neural stem cells into individuals that need the human neural stem cells. The human neural stem cell of the present invention has active effects for treating patients of neural system disorders and injuries, specifically for treating patients with a severe spinal cord injury, ischemic brain damage, epilepsy, and Alzheimer's disease, known to have no special treatment as of present and remain with permanent neurological aftereffects. Accordingly, the pharmaceutical composition containing the human neural stem cell of the present invention provides a novel method for treating neural system injuries.

### FIELD OF THE INVENTION

The present invention relates to a human neural stern cell, and to a pharmaceutical composition for the treatment of central or peripheral nervous system disorders or injuries using same. More particularly, the present invention relates to a human telencephalon-derived human neural stem cell effective in the treatment of nervous system disorders or injuries, and to a pharmacological composition for the treatment of nervous system disorders or injuries using same, to the use of the human neural stem cell for preparing therapeutic agents for the treatment of nervous system disorders or injuries, to a method for treating nervous system disorders or injuries, capable of administering an effective amount of the human neural stem cells into individuals who needs to be treated.

### DESCRIPTION OF THE RELATED ART

Neural stem cells are generally immature cells in the nervous system that can self-renew as undifferentiated, multipotency cells having a potential to differentiate into neurons and glial cells. Neural stem cells exist in various anatomical parts of mammalian fetal nervous systems, including in humans. Recently there have been reports that neural stem cells are also found in certain areas of human adult brain, and can form new neural cells by continuous proliferation in the specific brain area. In addition, neural stem cells are reported to differentiate from embryonic stem cells, which are immature cell or areas other than the nervous systems, such as bone marrow, skin, amniotic membrane, umbilical cord blood cell. However, the neural stem cell and nerve cell derived from these tissues are very rare and whether these cells can differentiate into truly functional neural cells is still unclear. In contrast, neural stem cells in the nervous system have been clearly shown to differentiate into nerve cells leading to increased research on finding the mechanism of stem cell proliferation and differentiation. There is increased interest for a novel cell and genetic therapy using the biological function of the neural stem cell for the treatment of primary neural disease, which is known to be impossible to regenerate and on secondary intractable nerves system injuries.

For the treatment of nervous system injuries, there was a search for various kinds of therapeutic drugs, proteins and neuro-nutritional factors and various methods are developed by assessing the therapeutic effect and neuroprotective reaction in vivo and ex vivo, but there is currently no report available. Also, no treatment method is available that is clinically proven to protect the nerve tissue or to regenerate it. Development of “small molecules”, such as therapeutic agents, neuro nutritional factors and compound materials are insufficient for the treatment of intractable nerve system disorders, but a cell therapy involving the induction of nerve regeneration by replacing the dead or functionally impaired nerve cells would be very promising. Due to recent advances in stem cell cytology, studies on human neural stem cell and its application as therapeutic agent is considered as the best solution to overcome the limitations and problems of safety and efficacy as well as limitation on using primary fetal tissue or primary cells, thus enabling gene therapy by using novel cells and stem cells.

For the application of human neural stem cell as a therapeutic agent, intensive basic and clinical research is required, as well as the problems related to the clinical application has to be solved as well. Further study is needed for regulation and control of human neural stem cell amplification and differentiation, long term safety testing, such as tumor suppression after transplant into human body, engraftment, migration, differentiation and integration of the donor stem cells into the host nervous system after transplantation, follow-up analysis on the nerve functions, achieving the desired stem cell transplantation goal by investigating the pathophysiology of intractable nerves system injuries, the difference and caution when applying the animal experimental data to clinical trials, the regulation of anti-inflammatory response and immune suppression response, pursuit of combined therapy between stem cells and other types of therapies (Alvarez-Buylla, et al. Nat Rev Neurosci 2:287, 2001; Flax, et al., Nat Biotech 16:1033, 1998; Gage, Science 287:1433, 2000; Lindvall, Nature 441:1094, 2006).

The incident of spinal cord injury increases as industry develops and the level of the human activity increases. In US, there are currently 1 million spinal cord injury patients and the frequency of the incident is 50 patients per 1 million people per year. That mean 12,000 new patients are occuring each year. In addition, most of the patients are young adults of ages 30 years-old or younger, having increased average survival period. It has been estimated that average of 9.7 million US dollars were spent as the medical cost per one year. In Korea, about 70,000 people are registered as disabled with spinal cord injury, and there is a strong correlation between the increase of car accidents and the increase in spinal cord injury patients. Spinal cord injury patients have severe motor, sensory and autonomic nerve damage, needing a long-term hospitalization and rehabilitation and absolute help form other people for their daily activities. These can cause great financial burden on society as well as on individuals, loss in human resources, and huge challenge in maintaining personal dignity and independence. However, the social welfare provided to help the spinal cord injury patients for their social activity and rehabilitation and maintaining independence is insufficient. The biological and medical research focusing on regenerating the injured spinal cord nervous system is still in its early phase.

In general, spinal cord injury is a central nervous system disorder, which is irreversible damage and therefore difficult to regenerate after the injury. In spite of the advance in the field of the treatment of spinal cord injury and rehabilitation methods, the treatment of the basic cause of the injury is impossible since nerve tissue does not regenerate. Therefore, surgical procedures and drug therapy to prevent the secondary spinal cord injury is the only available therapeutic method in clinical settings. In the case of acute spinal cord injury, methylprednisolone can be administrated, but the efficacy of this drug is not clear and leads to complications, therefore many countries have banned the use of this drug. Drugs such as manosialoganglioside sodium [GM-1 ganglioside], naloxone, and tirilazad etc.) showed effects on spinal cord injury in vitro and were used for clinical trials but the result was unclear. As of today, there is no neuro protective drug that is US FDA approved to be used in clinical trials (Ducker et al., Spine 19:2281, 1994; Hurlbert, J Neurosurg 93:1, 2000; Short, et al., Spinal Cord 38:273, 2000; McDonald, et al., Lancet 359:417, 2002).

Recently, research in the regenerative medicine field is gaining attention in the hope of developing a therapy for spinal cord injury. At the area of spinal cord injury various myelin associated molecule and glial scar-associated extracellular matrix proteins interacts to inhibit the regeneration of injured neuronal axons. There are several reports using various cells and tissues in animal models and in clinical trials to induce the regrowth and regeneration of the damaged spinal cord nerve axons (Bradbury et al., Nature 416:636, 2002; Bregman et al., Nature 378:498, 1995; GrandPre, et al., Nature 417:547, 2002; Bunge, Neuroscientist 7:325, 2001; Cheng et al., Science 273:510, 1996; Coumans et al., J Neurosci 21:9334, 2001; Keyvan_Fouladi et al., J Neurosci 23:9428, 2003; Rossignol et al., J Neurosci 27:11782, 2007).

Lately, there were reports on animal experiments where stem cells were transplanted into the spinal cord injury area to induce the differentiation into nerve cells, induction of the nerve axons and remyelination of axons. When mouse and human embryonic stem cells were induced to differentiate into nerve cells and transplanted, there was an improvement in the neural function, but the problem with tumor formation and ethical issues regarding the use embryonic stem cell has to be solved (McDonald et al., Nat Med 5:1410, 1999; Keirstead et al., J Neurosci 25:4694, 2005). The problem of immune rejection response and ethical issues can be avoided by using bone marrow stromal stem cells but the trans-differentiation of neural stem cells into functional neural stem cell is still not clear (Terada, et al., Nature 416:542, 2002; Ying et al., Nature 416:545, 2002; Alvarez-Dolado et al., Nature 425:968, 2003). Even though there are reports that stem cells do not differentiate into nerve cells when transplanted in to the spinal cord injury area (Hofstetter, et al., PNAS 99:2199, 2002), there are reports on the possible induction of regeneration of axons in the injured spinal cord by the donor cells (Ankeny et al., Exp Neural 190:17, 2004). The neural stem cell derived from the rodent or human central nervous systems can differentiate into functional nerve cells in the area of the spinal cord injury, does not have the possibility of forming tumors and showed improvement in the neural functions (Cummings et al., PNAS 102:14069, 2005; Hofstetter et al., Nat Neurosci 8:346, 2005; Iwanami et al., J Neurosci Res 80:182, 2005; Karimi-Abdolrezaee et al., J Neurosci 26:3377, 2006; Ogawa et al., J Neurosci Res 69:925, 2002; Teng et al., PNAS 99:3024, 2002; Yan et al., PLos Medicine 4:e39, 2007). Recent papers have published that in spinal cord injury, the injuries of neuroglial cells in the white matter is more critical than the injuries of the neurons in the white matter. There were reports on animal experiments where glial-restricted progenitor cells (GRPs) or oligodendrocyte precursor cells (OPCs) were transplanted into the animals (Bambakidis et al., Spine J 4:16, 2004; Cao et al., J Neurosci 25:6947, 2005; Han et al., Glia 45:1, 2004; Herrera et al., Exp Neurol 171:11, 2001; Hill et al., Exp Neurol 190:289, 2004; Mitsui et al., J Neurosci 25:9624, 2005). In summary, different types of stem cells are reported to improve the neural function in animals transplanted with the cells. However, it is still unclear which stem/progenitor cell is the ideal donor for the clinical application. Therefore, in order to achieve better therapeutic effects, different therapy goals and steps should be establishing by understanding the pathophysiology of spinal cord injury and also a combined approach is required by mixing the stem/progenitor cells with other types of therapies.

Stroke is the second cause of death in Korea, and number one cause of death as a single disorder, which is a very important public health issue. In adults, there are 150,000 new incidents of stroke patients per each year. The number of incidents of hypoxic-ischemic brain injury in fetal and neonatal by prenatal asphyxia is 2-4 out of 1000 full term live birth babies (2000 new patient babies per year) and occurs 60% in very low birth weight premature babies (3000-9000 new patient babies per year). About 10-60% of hypoxic-ischemic brain injury babies die during the neonatal period, showing a very high death rate. Out of the babies survived, 25% of the babies suffer severe permanent long-term effects, such as cerebral palsy, delayed mental development, learning disorder and epilepsy. Together with stroke in adults, this is a major nervous system disorder that causes a burden in public health care systems as well as social and economical aspects. Currently, no therapeutic method is available for clinically improving the hypoxic-ischemic brain injury. The only therapeutic method available is a conservative therapy method, which cannot prevent or stop the brain damage in progress (Rogers M C, Nichols D G (Eds). Rogers' Textbook of Pediatric Intensive Care. 4th Edition. Philadelphia, Lippincott Williams & Wilkins, 2008, pp 810-825; Roach et al., Stroke 39:2644, 2008; van Bel and Groenendaal, 2008 Neonatology 94:203).

Epilepsy is one of the most common nervous system disorders, where 3-5% of the population experiences a convulsion symptom once in their life, and 0.5-1% of the population are epilepsy patients showing repeated seizures. Most of the patients can be treated when administered with anti-convulsion drugs, but 20% of primary generalized epilepsy patients and 35% of partial epilepsy patients are a refractory epilepsy patient who does not response to the drug therapy. A surgical method of removing part of the brain can be considered to replace the anti-convulsion drug therapy, but only 50% (⅔ when patients were carefully selected according to the surgery indicant) of the patients showed complete disappearance of epilepsy symptoms and rest of the patients showed the effect of reduction in epilepsy frequency or weakening of the intensity. Some patients are not suitable for the surgery because of the high risk for loss in brain function. Neonatal or infant patients with catastrophic epilepsy syndromes show diffusion, bilateral or multifocal epilepsy in brain wave of EEG, therefore being unsuitable for surgery. Even though they are suitable for a surgery, the brain tissue damaged by multilobar resection or hemispherectomy can cause neurological deficits. Therefore, despite the vast number of therapy methods developed, the total number of refractory epilepsy patients is larger than the total number of patients with intractable nervous system disorders, such as different types of brain tumors, multiple sclerosis, muscular dystrophy, spinal cord motor nerve disorder and Guillain-Barre syndrome. For this reason, developing a novel therapeutic method for refractory epilepsy is very important in the aspect of public health. A therapeutic method for the treatment of epilepsy can be developed by transplanting neural stem cells into the regions that induce epilepsy, initiates the convulsion, and decreases the function. This transplant would replace the nerve cells that are injured or have decreased function, reconstruct the neruociruits, and regulate the hyper-excitatory state. The development of therapeutic method that inhibits the epileptic seizures and improves the nerve functions would be a revolutionary and fundamental approach for the treatment of epilepsy (Rakhade and Jensen, Nat Rev Neurol 5:380, 2009; Marson et al., Clin Evid pil: 1201, 2009; Banerjee et al., Epilepsy Res 85:31, 2009; Jacobs et al., Epielepsy Behav 14:438, 2009).

Alzheimer's disease is progressive brain disease usually observed in elderly people, and which is the main cause of the senile dementia. About 50% of senile dementia patients are Alzheimer's disease patients (vascular dementia 30-40%, metabolic dementia 10-20%, unknown reason 50%). The onset rate and the prevalence rate increases with age, where the rate doubles every 5 years after the age of 65. The prevalence rate of the Alzheimer's is 1% in 60-64 years-old, 2% in 65-69 years-old, 4% in 70-74 years-old, 8% in 75-79 years old, 16% in 80-84 years-old and up to 35-40% in over 85 years-old. Approximately 10% of the elderly people suffer Alzheimer's disease. The onset rate 2.3% in 75-79 years-old, 4.6% in 80-84 years-old and up to 8.5% in over 85-89 years-old. In year 2000, the population o f the elderly people above 65 years-old was 7% in Korea. By year 2022, the population of the elderly people above 65 years-old would be over 14%, and becoming an aging society. There would be a sharp increase in the number of Alzheimer's disease patients as well. In year 2007, the number of dementia patients in Korea was 399,000, which 8.3% of the total elderly people (4.8 million), indicating a 41.4% increase compared to 7 years ago (at year 2000; 282,000). The rate of domestic dementia patients are the highest compared to other countries; Japan (3.8%), UK (2.2%), USA (1.6%), Spain (1.0%). The rate of aging would increase faster than any other countries, so the there would be an increase of dementia patients from 461,000 (8.6% of elderly population) to 580,000 (9%) by year 2015. There is a great financial loss due to dementia last year. The direct medical cost was 300 million dollars but when combining with the indirect cost, it will be estimated to be up to several trillion dollars. In US, there are 5.2 million Alzheimer's disease patients, where 500, 000 patients are younger than 65 years old. Especially, it is estimated that 18% of the baby boomers would develop Alzheimer's disease, which would be about 14 million patients. In US, 70% of the patients are taken care of by their family members at their homes. In last year alone, the number of caregivers was 10 million people, the time spent was 8.4 billion hours and the total coast was trillions of dollars. Recently, some therapeutic drugs have been developed to improve the symptoms and delay the progress of the disease but which does not treat the main cause. Increase in elderly population having Alzheimer's disease will causes a burden in the public health care systems as well as in social and economical aspects. The development of therapeutic method for the treatment of Alzheimer's disease using neural stem cells would be a revolutionary and fundamental approach for the treatment of progressive nerve system disorders (Minati et al., Am J Alzheimers Dis Other Demen 24:95, 2009; Ziegler-Graham et al., Alzheimers Dement 4:316, 2008; Kalaria et al., Lancet Neurol 7:812, 2008).

### DETAILED DESCRIPTION OF THE INVENTION

### Technical Problem

The present inventors have made intensive research to develop an effective method for the treatment of nervous system disorders or injuries, such as spinal cord injury. As a result, the inventors have discovered that human telencephalon-derived human neural stem cells can be used as a safe and effective method for treating the nervous system disorders or injuries, thus completed the present invention.

Accordingly, it is an object of this invention to provide a human neural stem cell assigned with depository number KCTC11370BP and a pharmaceutical composition comprising the human neural stem cell for the treatment of nervous system disorders or injuries.

### Technical Solution

In order to achieve above mentioned objective, the present invention provides a human neural stem cell assigned with depository number KCTC11370BP.

It is another object of this invention to provide a pharmaceutical composition comprising the human neural stem cell for the treatment of nervous system disorders or injuries.

It is still another object of this invention to provide a use of the human neural stem cell for preparing therapeutic agents for the nervous system disorders or injuries.

It is further object of this invention to provide a method for treating nervous system disorders or injuries, capable of administering an effective amount of the human neural stem cells into individuals who needs treatments.

Hereinafter, the present invention will be described in detail.

The human neural stern cell of the present invention was nerve tissue derived from human fetal telencephalon legally aborted at 13-gestational-week, cultured into genetically unmodified primary human stem cell using specific growth factors, and was confirmed to have stem cell characteristics by in vitro experiments. The safety and efficacy of the human stem cell was assessed and verified by transplanting into spinal cord injury model before its clinical use. The human stem cell deposited to Korea Research Institute of Bioscience and Biotechnology on Jul. 24, 2008 under depository number KCCM11370BP.

The term “stem cell” used herein refers to master cells that can reproduce indefinitely to form the specialized cells of tissues and organs. Stem cells are developmental pluoripotent or multipotent cells. Stem cells can divide to produce two daughter stem cells, or one daughter stem cell and one progenitor (“transit”) cell, which then proliferates into fully differentiated and mature cells in tissue.

The term “multipotent cells” used herein refers to cells that have the capacity to develop into any subset of approximately 260 cell types in the mammalian body. Unlike pluripotent cells, multipotent cells do not have the capacity to form all of the cell types.

The term “differentiation” used herein refers to a phenomenon in which the structure or function of cells is specialized during the division, proliferation and growth thereof, that is, the feature or function of cell or tissue of an organism changes in order to perform work given to the cell or tissue. Generally, it refers to a phenomenon in which a relatively simple system is divided into two or more qualitatively different partial systems. For example, it means that a qualitative difference between the parts of any biological system, which have been identical to each other at the first, occurs, for example, a distinction, such as a head or a body, between egg parts, which have been qualitatively identical to each other at the first in ontogenic development, occurs, or a distinction, such as a muscle cell or a nerve cell, between cells, occurs, or the biological system is divided into qualitatively distinguishable parts or partial systems as a result thereof.

The term “cell therapeutic agent” used herein refers to a drug used for the purpose of treatment, diagnosis and prevention, which contains a cell or tissue prepared through isolation from man, culture and specific operation (as provided by the US FDA). Specifically, it refers to a drug used for the purpose of treatment, diagnosis and prevention through a series of behaviors of in vitro multiplying and sorting living autologous, allogenic and xenogenic cells or changing the biological characteristics of cells by other means for the purpose of recovering the functions of cells and tissues. Cell therapeutic agents are broadly divided, according to the differentiation level of cells, into somatic cell therapeutic agents and stem cell therapeutic agents, and the present invention relates to the stem cell therapeutic agents.

The neural stem cell may be derived from human fetal telencephalon. Preferably, cells derived from human fetal brain tissue may be cultured in a medium supplemented with neural stem cell growth factors (see example 1). The neural stem cell growth factor may be bFGF (fibroblast growth factor-basic), LIF (leukemia inhibitory factor) and heparin. Preferably, 20 ng/ml bFGF, 10 ng/ml LIF and 8 pg/ml heparin may be used.

The human neural stem cell may be proliferated and cultured according to the conventional techniques. The neural stem cell is cultured in a culture medium supporting the survival and the proliferation of the object cell type. Preferably, culture medium containing free amino acid instead of serum for nutrition may be used. Preferably, culture medium may be added with supplement developed for continuous neural cell culture. For example, the supplement is N2 and B27 manufactured by Gibco. Preferably, the medium is replaced by observing the condition of the cell and the culture medium. More preferably, the neural stem cells are subcultured when the cells continue to proliferate and form cluster of cells called neurospheres. Subculture may be performed every 7-8 days.

Preferably, the neural stem cells according to the present invention are cultivated as follows. N2 or B27 additives (Gibco), neural stem cell growth inducing cytokines (e.g., bFGF, EGF, LiF, etc.) and heparin are added to a culture medium with known compositions (e.g., DMEM/F-12 or Neurobasal medium). In general, serum is not added. In the medium, the neural stem cells grow into neurospheres. About half of the medium is replaced with fresh one every 3 to 4 days. When the number of the cells increases, the cells are dissociated every 7 to 8 days mechanically or using trypsin (0.05% trypsin/EDTA, Gibco). Then, the cell suspension is plated on a new plate and cultivated further using the same medium (Gage et al., PNAS 92(11): 879, 1995; McKay, Science, 276:66, 1997; Gage, Science, 287:1433, 2000; Snyder et al., Nature, 374:367, 1995; Weiss et al., Trends Neurosci., 19:387, 1996).

The neural stem cells of the present invention may be differentiated into various nerve cells according to the methods known in the related art. In general, differentiation of the cells is carried out using a nutrient medium including an adequate substrate or differentiation reagent but without including a neural stem cell growth inducing cytokine. A preferred substrate is a solid surface coated with cationic charges, for example, poly-L-lysine and polyomithine. The substrate may be coated with extracellular matrix components, for example, fibronectin and laminin. Other allowable extracellular matrix includes Matrigel. In addition, a mixture of poly-L-lysine with fibronectin or laminin, or a combination substrate thereof may be used.

Adequate differentiation reagent includes a variety of growth factor, e.g., epidermal growth factor (EGF), transforming growth factor-α (TGF-α), all types of fibroblast growth factors (FGF-4, FGF-8 and bFGF), platelet-derived growth factor (PDGF), insulin-like growth factors (IGF-I and others), high-concentrated insulin, bone morphogenetic proteins (particularly, BMP-2 and BMP-4), retinoic acid (RA) and ligands that bind with the gpl30 receptor (e.g., LIF, CNTF and IL-6), but is not limited thereto.

The neural stem (mils of the present invention may be preserved in frozen status (cryopreservation) for long-term storage. In general, cryopreservation is performed as follows. When a sufficient quantity of neural stem cells is acquired through repeated subculturing, the resultant neurospheres are dissociated mechanically or using trypsin to obtain a single cell suspension. Then, the cell suspension is mixed with a cryopreserving solution consisting of 20-50% fetal bovine serum (Gibco), 10-15% DMSO (Sigma) and cell culture medium, and moved into a freezing vial (NUNC). The cells mixed with the cryopreserving solution are immediately transferred to a freezer of −70° C., after kept at 4° C., and moved to a liquid nitrogen tank after at least 24 hours for long-term storage (Gage et al., PNAS, 92(11): 879, 1995; McKay, Science, 276:66, 1997; Gage, Science, 287:1433, 2000; Snyder et al., Nature, 374:367, 1995; Weiss et al., Trends Neurosci, 19:387, 1996).

The cryopreserved neural stem cells of the present invention may be thawed by the method known in the related art. The cryopreserved cells may be thawed by immersing the freezing vial in a water bath of 37° C. and shaking slowly. When about half of the cells in the freezing vial are thawed, the cell suspension is moved into a conical tube containing a neural stem cell medium, which is warmed to 37° C. When all the cell suspension is transferred, centrifuge is carried out and the supernatant removed. The precipitated cell pellet is cautiously floated into the neural stem cell medium. Then, the cell suspension is moved to a 60 mm cell culture plate. Subsequently, neural stem cell growth inducing cytokine is added to the medium, and cultivation is carried out in a 5% CO₂ incubator at 37° C.

Further, the present invention provides a pharmaceutical composition comprising the human neural stem cell for the treatment of neural system disorders or injuries.

As used herein, the term “treatment” refers to alleviation of symptoms, diminishment of extent of disease, stabilized state of disease, delay or slowing of disease progression, amelioration or palliation (whether partial or total), of the disease state, and remission. “Treatment” can also mean an improved state as compared to expected improved state if not receiving treatment. “Treatment” includes preventive measures as well as treatment measures. Necessary treatment conditions are when already in the disease stated when disease needs to be prevented. The term “palliation” used herein means that the extent or undesirable clinical manifestations of a disease state are lessened and/or the time course of the progression is slowed or lengthened, as compared to the disease in the absence of the substance and/or composition of the present invention. In general, treatment is administration of the human neural stem cell into injured neural system. The neural system is brain, telencephalon and peripheral nervous system.

The human neural stem cell is administered to the desired tissue part via direct transplantation or migration method, thus helping the injured neural system to regenerate or recover its function. For instance, the human neural stem cell was injected into the injured nerve area of the disorder to be treated. Transplantation was performed using single cell suspension or small cell clusters of 1×10⁵-1.5×10⁵/μl cell density (U.S. Pat. No. 5,968,829).

The human neural stem cell can be provided as a pharmaceutical composition to be administered in human. The pharmaceutical composition may comprise a pharmaceutically acceptable carrier. The term “pharmaceutically acceptable” used herein refers to being non-toxic to cells or human when exposed by the composition. The pharmaceutically acceptable carrier may be conventional one including, but not limited to buffer, preservatives, epidural, solubilizer, isotonic solution, stabilizer, spray, suspending agent and lubricant. According to the conventional techniques known to those skilled in the art, the pharmaceutical compositions of this invention can be formulated with pharmaceutical acceptable carrier and/or vehicle as described above. For example, the formulation for parenteral administration may be presented in unit dose ampoules or multi-dose containers. For general principles in medicinal formulation of the pharmaceutical composition, the reader is referred to Cell Therapy: Stem Cell Transplantation, Gene Therapy, and Cellular Immunotherapy, by G. Morstyn & W Sheridan eds, Cambridge University Press, 1996; and Hematopoietic Stem Cell Therapy, E. D. Ball, J. Lister & P. Law, Churchill Livingstone, 2000. The pharmaceutical composition may be packaged in a suitable container for the purpose, for example, to regenerate the injured nervous systems.

Further, the present invention provides a use of the human neural stem cell for preparing therapeutic agents for the treatment of nervous system disorders or injuries. In addition, the present invention provides a method for treating nervous system disorders or injuries by administering an effective amount to a subject.

The human neural stem cell and the effect of the neural stem cell in present invention is same as described above. As used herein, the term “effective amount” refers to an amount for achieving the treatment effect for nervous system disorders or injuries in administered subjects. As used herein, the term “subject” is to include any member of the mammalian class. In particular, the mammalian class includes a human.

The human neural stem cell can be administered until the desired effect is achieved, and can be administered via various delivery routes according to the conventional techniques known to those skilled in the art.

The nervous system disease and injury which may be applied with the pharmaceutical composition, the use and the method for treatment includes, but not limited to spinal cord injury, Parkinson's disease, stroke, amyotrophic lateral sclerosis, motor nerve injury, traumatic peripheral nerve injury, ischemic brain injury, neonatal hypoxic-ischemic brain injury, cerebral palsy, epilepsy, refractory epilepsy, Alzheimer's disease, congenital metabolic nervous system disorders, and traumatic brain injury.

In an example of this invention, the nerve cells derived from legally aborted human fetal telencephalon was cultured into human neural stem cell using growth factors.

In another example of this invention, the safety and efficacy was assessed by transplanting the human stem cell into spinal cord injury model. As a result, the human neural stem cell was proven to be safe and efficient, hence no specific toxicity was detected and was capable of the treatment of the spinal cord injury.

In still another example of this invention, the human neural stem cell was transplanted into patients with spinal cord injury. The patients received physical and occupational therapy commonly used in spinal cord injury patients and their improvement was monitored. As a result, in total of 17 clinical cases, 1 patient out of 15 ASIA-A patients improved to ASIA-B, 2 cases improved to ASIA-C, 2 patients out of 2 ASIA-B patients improved to ASID-D, indicating 29% of clinical improvement in ASIA levels in complete spinal cord injury patients. Especially, according to the surgical opinion, 3 cases in ASIA-A group had severe spinal atrophy to pect any clinical improvements. Therefore, when excluded, 25% of ASIA-A patients showed improvement after human stem cell transplant and 36% of improvement in complete spinal cord injury patients after human stem cell transplant. In addition, 75% of ASIA-A patients and over 84% of the complete spinal cord injury patients (14 out of 17 cases) showed improvement in motor function.

In another example of this invention, the safety and the efficacy of the human neural stem cell was analyzed by transplanting human stem cells into the neonatal hypoxic-ischemic animal model. As a result, the human neural stem cell was proven to be safe and efficient, hence no specific toxicity was detected and was capable of the treatment of the hypoxic-ischemic injury.

In another example of this invention, the safety and the efficacy of the human neural stem cell was analyzed by transplanting human stem cells into the refractory epilepsy animal model. As a result, the human neural stem cell was proven to be safe and efficient, hence no specific toxicity was detected and was capable of the treatment of the refractory epilepsy.

In another example of this invention, the safety and the efficacy of the human neural stem cell was analyzed by transplanting human stem cells into the Alzheimer's disease animal model. As a result, the human neural stem cell was proven to be safe and efficient, hence no specific toxicity was detected and was capable of the treatment of the Alzheimer's disease

### Advantageous Effects

Therefore, the human neural stem cell of the present invention has active effects on the treatment of patients with nervous system disorders or injuries, particularly on the treatment of patients with spinal cord injury, where currently no treatment is available to reverse the permanent neurological deficits, Parkinson's disease, stroke, amyotrophic lateral sclerosis, motor nerve injury, traumatic peripheral nerve injury, ischemic brain injury, neonatal hypoxic-ischemic brain injury, cerebral palsy, epilepsy, refractory epilepsy, Alzheimer's disease, congenital metabolic nervous system disorders, and traumatic brain injury. In addition, the pharmaceutical composition of the present invention comprising the human neural stem cell provides a novel method for treating neural system injuries.

### BRIEF DESCRIPTION OF THE DRAWINGS

FIG. 1 is an image showing the engraftment of human neural stem cell in spinal cord migrating towards the injured area and its surroundings (red: area showing the engraftment of human neural stem cell staining immunopositive for human-specific nuclei antigen (hNuc; Chemicon, Temecula, Calif.), green: immunopositive staining of neurofilament (NF; Sternberger, USA) in host spinal cord neurite.

FIG. 2 is fluorescent images showing the detection of neural stem cell differentiated into neurons, astrocytes and oligodendrocytes or undifferentiated neural stem cell (A; detection of early neuronal marker TUJ1 (β-tubulin III, Covance) expression (arrow), B; detection of astrocyte marker GFAP (glial fibrillary acidic protein, DAKO) expression (arrow), C; detection of oligodendrocytes marker CNPase (2,3-cyclic nucleotide-3-phophohydrolase, Chemicon) expression (arrow), D; detection of undifferentiated human neural stem cell maker hNestin (human nestin, Chemicon) expression (arrow).

FIG. 3 is fluorescent images showing the engraftment of human neural stem cell migrated towards the area of cerebral infarction. (red: area showing the engraftment of human neural stem cell staining immunopositive for human-specific nuclear matrix (hNuMA; Calbiochem, Germany). The transplanted neural stem cell differentiated into neurons, astrocytes and oligodendrocytes (green: detection of neuronal marker Neurofilament (NF, Steinberger, USA) expression, detection of oligodendrocytes marker Myelin Basic Protein (MBP; DAKO, Carpinteria, Calif.), detection of astrocyte marker glial fibrillary acidic protein (GFAP; DAKO, Carpinteria, Calif.) expression). The co-expression of red and green is detected by a yellow color.

FIG. 4 is images showing the expression of neurotransmitters in transplanted human neural cells when differentiation into neuronal cells (red: human-specific nuclear matrix (hNuMA; Calbiochem, Germany) immunopositive human neural stem cell, green: detection of the glutamatergic neuronal marker, glutamate (Glut; Sigma, Saint Louis, Mo.); detection of GABAnergic neuronal marker, γ-Aminobutyric acid (GABA; Sigma, Saint Louis, Mo.); detection of Choline acetyl transfers (Chat; Chemicon, Temecula, Calif.), a marker for cholinergic neuron; detection of Synapsin-1 (Syn-1; Chemicon, Temicula, Calif.), a marker of synapse formation).

FIG. 5 is a graph representing the neurological test in a hypoxic-ischemic animal model transplanted with human neural stem cell (hNSC) or the H-H buffer (vehicle). The animals were monitored every 2 weeks when they were 3 weeks old until 11 weeks old.

FIG. 6 is a graph representing the acquisition of spatial memory and long-term memory in an animal model with the hypoxic-ischemic animal model transplanted with human neural stem cell (hNSC) or the H-H buffer (vehicle). The spatial learning session was performed for 6 days and the probe test was performed on day 7 and the time spent in the quadrant was measured (goal quadrant spent time).

FIG. 7 is images showing the engraftment of transplanted human neural stem cell migrating towards the transplanted area and its surroundings (green: BrdU immunopositive human neural stem cell, red: Tuj1 immunopositive neuronal cell. The cell merged with green and red colors are shown in yellow.

FIG. 8 is images showing the differentiation of human neural stem cell into GABA expressing neuronal cells or oligodendrocytes but into astrocytes. (A; BrdU positive donor cells shown in green expressing GABA are shown in red color. The green and red cells that immerged are shown as yellow or orange color, B; BrdU positive donor cells shown in green expressing oligodendrocyte marker APC-CC1 [adenomatous polysposis coli clone CC1, Abcam, UK] and shown in red color. Green and red cells that immerged are shown as yellow or orange color, C; BrdU positive donor cells shown in green did not press the red colored astrocytes maker, GFAP [glial fibrillary acidic protein, DAKO].

FIG. 9 is graphs representing human neural stem cell transplanted into kindling model which is an animal model for refractory epilepsy. The inhibitory effect was video monitored (FIG. 9A) and analyzed by EEG recording device (FIG. 9B). Racine's scale (scale 1; facial movements only, scale 2; facial movements and head nodding, scale 3; facial movements, head nodding, and forelimb clonus, scale 4; facial movements, head nodding, forelimb clonus, and rearing, scale 5; facial movements, head nodding, forelimb clonus, rearing, and falling, scale 6; facial movements, head nodding, forelimb clonus, and a multiple sequence of rearing and falling) was used to measure the seizure intensity. EEG showing spikes of 1 Hz or higher frequencies were considered as an elliptical activity and the duration time was analyzed (Y Kreano, et al, Epilepsia 2005; 46:1561, LW, et al., Eur J Phamacol. 1989; 163;1). Asterisk denotes the value that is statistically significant (p<0.05).

FIG. 10 is images showing human neural stem cell transplanted into APPsw transgenic mice migrated and engrafted from lateral ventricle area to cerebral cortex, hippocampus and corpus callosum. (red color by immunofluorescent staining: human specific nuclear matrix (hNuMA; Calbiochem, Germany), human specific heat shock protein 27 (hHsp27; Stressgen, Ann Arbor, Mich.).

FIG. 11 is images showing the number and distribution of microglia cells in APPsw in APPsw transgenic mice transplanted with human neural stem cell (APP-hNSC) group and APPsw transgenic mice transplanted with H-H buffer transplanted group (APP-vehicle) using microglia cell markers (green: CD11b [AbD Serotec, UK] and F4/80 [AbD Serotec, UK]) under immunofluorescent microscopy. The human neural stem cell transplanted group showed significant difference in reduction of microglia cell markers compared to H-H buffer transplanted group.

FIG. 12 is a series of graphs representing the acquisition of spatial memory and long-term memory in four experimental groups; APP-hNSC; APPsw transgenic mice transplanted with human neural stem cell, APP-vehicle; APPsw transgenic mice transplanted with H-H buffer, Wild-hNSC; wild type mice transplanted with human neural stem cell, Wild-vehicle; wild type mice transplanted with H-H buffer. The four experimental groups did not show any significant difference in latency to find a hidden platform when tested for six days (FIG. 12A). Comparison of the escape latency on day 7 showed a statistically significant improvement in the escape latency of APPsw transgenic mice transplanted with human neural stem cell when compared with APPsw transgenic mice transplanted with H-H buffer on day 7 of the test.

### DETAILED DESCRIPTION OF THIS INVENTION

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Culture of Human Neural Progenitor Cells

### 1.1. Isolation of the Brain Tissue

Human fetal tissue from cadavers by miscarriage at 13 weeks of gestation was obtained with full patient consent and the approval of the IRB (Institution Review Board) of Yonsei University College of Medicine, Severance Hospital. The methods of acquisition conformed to the guidelines of Ministry of Health and Welfare, Ministry of Education, Science and Technology and Yonsei Severance Hospital. The fetal tissue was washed in an ice-cold H-H buffer solution (Hank's balanced salt solution, 1× [GIBCO]+HEPES, 10 mM [GIBCO] in ddH2O, pH 7.4) and CNS tissue was dissected under microscope. Meninges and blood vessels were removed and telencephalon was separated. The tissue was dissected into 1×1 mm size pieces in a petri dish. The supernatant was removed after centrifugation at 950 rpm for 3 min. The tissue was washed again with H-H buffer solution, and centrifuged. This procedure was repeated for three times. The supernatant was discarded after the last centrifugation step, then 5 ml of 0.1% trypsin (Gibco) and DNase I (Roche, 1 mg/dl) were added and mixed with the remaining pellet This mixture was incubated at 37° C. for 30 min before adding 5 ml of H-H buffer solution containing trypsin inhibitor (T/I, Soybean, Sigma, 1 mg/ml). The pellet was slowly dissociated into a single cell level using serologic pipette (Falcon). Then the sample was centrifuged, and the supernatant was discarded. The remaining cell pellet was washed with H-H buffer solution. The cell pellets were centrifuged again and the supernatant was discarded.

### 1.2 Proliferation of Neural Stem Cell

The brain tissue cell pellet from example 1.1 was slowly mixed with 10 ml of N2 medium (D-MEM/F-12 [98% volume (v)/volume(v)]+N2 supplement [1% v/v]+Penicillin/Streptomycin [1% v/v]; GIBCO). About 4×106-6×106 cells were transferred to tissue culture treated 100 mm plate (Corning). Twenty ng/ml of bFGF (recombinant Human fibroblast growth factor-basic (R&D) was used as neural stem cell growth factor, then 10 ng/ml LIF (recombinant human leukemia inhibitory factor, Sigma) and 8 μg/ml of heparin (Sigma) were each added, vortexed and incubated at 37° C. in 5% CO2 in air. After 24 hrs, 5 ml of medium was discarded the replace with 5 ml of fresh N2 medium. Twenty ng/ml of bFGF, 10 ng/ml LIF and 8 μg/ml of heparin (Sigma) were added and further incubated. The medium was replaced every 3-4 days by monitoring the medium and the cell conditions. Half of the medium was replaced with the new medium then growth factors were added.

### 1.3. Subculture

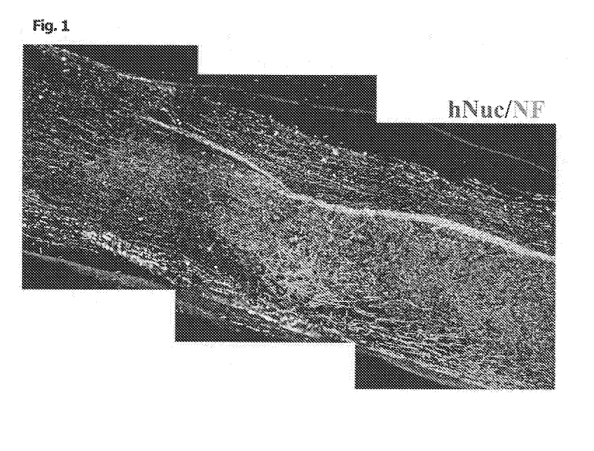

Cells were subcultured every 7-8 days, when the undifferentiated neural stem cell continues to proliferate and forms a cluster of cells called neurospheres according to example 1.2 (FIG. 1). Subculture was performed as follows: The culture medium was removed from the cell culture plate. The cells were treated with 2 ml of 0.05% trypsin/EDTA (T/E, Gibco), then incubated at 37° C. in 5% CO2 in air for 2 min 30 sec. To inhibit the activity of trypsin, 2.5 ml of trypsin inhibitor (T/I, Soybean, Sigma, 1 mg/ml) was added and mixed. Fifteen ml of cell suspension was transferred to the comical tube (Falcon). The supernatant was discarded after centrifugation. Cells were resuspended with 3 ml of N2 medium and the neurospheres were dissociated into single cells using serologic pipette. The cell number was counted and the cell suspension with the cell density of 4×10⁶-6×10⁶ were transferred to the new cell culture plate. Fresh N2 culture medium was added to a final volume of 10 ml. Twenty ng/ml of bFGF, 10 ng/ml LIF and 8 μg/ml of heparin (Sigma) were added and further incubated in 5% CO2 incubator.

### 1.4. Cryopreservation

Some of the neural stem cell was cryopreserved after enough number of neural stem cell were obtained according to the method described in example 1.3. The cryopreservation was performed as follows. The neurospheres were treated with 0.05% trypsin/EDTA (T/E, Gibco) to dissociate then transferred to a 15 ml falcon tube. The cells were washed with 8 ml of H-H buffer solution. The supernatant was discarded after centrifugation. The cell pellets were gently resuspended with 4° C. cryopreservation solution (N2 medium [40% v/v+FBS [50% v/v]+DMSO [10% v/v, Sigma]). Each freezing vial (NUNC) was aliquoted with 1.8 ml of the cell suspension. On average, the cells collected from one 10 mm cell culture plate were aliquoted into 3-4 freezing vials. The vials were kept in an ice bucket before being stored at −70° C. deep freezer for at least 24 hrs. The vials were placed in a liquid nitrogen tank for longer storage.

### 1.5 Thawing the Cryopreserved Cells

The cryopreserved cells were thawed by incubating in a 37° C. water bath with gentle agitation. When half of the cells are thawed, the cell suspension was transferred into the comical tube containing 10 ml of N2 medium preheated to 37° C. The supernatant was discarded after centrifugation. The cell pellets were carefully resuspended with 5 ml of N2 medium and transferred to 60 mm cell culture plate. Next, 20 ng/ml of bFGF, 10 ng/ml LIF and 8 μg/ml of heparin (Sigma) were added and further incubated at 37° C. in 5% CO2 incubator. Once the cells grew and formed neurospheres, they were subcultured again according to the method described in example 1.3. When the cells had grown for 10 days, the cells were transferred to a 10 mm cell culture plate.

### Example 2

Transplantation of Human Neural Stem Cell into Mice and Analysis of Its Effect

The effect of human neural stem cell on regeneration after spinal cord injury was analyzed by transplanting the human neural stem cell into the spinal cord injury animal model. For spinal cord injury, NYU (New York University) impact model was used. Adult Sprague-Dawley rats (300-350 mg in body weight) were anesthetized and a laminectomy was done at T9-10 level (thoradc spine), and the dorsal surface of the cord was subjected to a weight-drop impact, using a 2 mm diameter, 10-gram weight dropped at a height of 25 mm to induce a moderate contusive spinal cord injury (Basso et al., J Neurotrauma 1995; 12:1, Liu et al., J Neurosci 1997; 17:5395). Seven to eight days after inducing the spinal cord injury, 10 μl (4×10⁴) of the human neural stem cell prepared were transplanted using a glass micropipette. To suppress the immune rejection response, the cell transplanted group and the H-H buffer injected control group were injected with immune suppressor cyclosporine (10 mg/kg) every day via intraperitoneal injection, starting one day before the cell transplant and continuing until 12 weeks post-transplant.

The mice spinal cord section was analyzed 2, 4, 6 and 12 weeks after the cell transplant. As shown in FIG. 1, human neural stem cell immunopositively stained as red color for human-specific nuclei antigen (hNuc; Chemicon, Temecula, Calif.) were detected in the region of spinal cord injury and was also shown to have migrated and engrafted into the surrounding area. Following the engrafted donor cell region was a long tension of green colored immunopositive neurofilaments (NF; Sternberger, USA) of host spinal cord neurite. This suggests that the donor cells migrated and engrafted in the cell transplanted region and the neighboring area of the L1-2 segment of the spinal cord. Near the engrafted donor cells, there was a tension of host spinal cord neurite, suggesting that the transplanted donor cells have induced the regeneration of host spinal cord neurite.

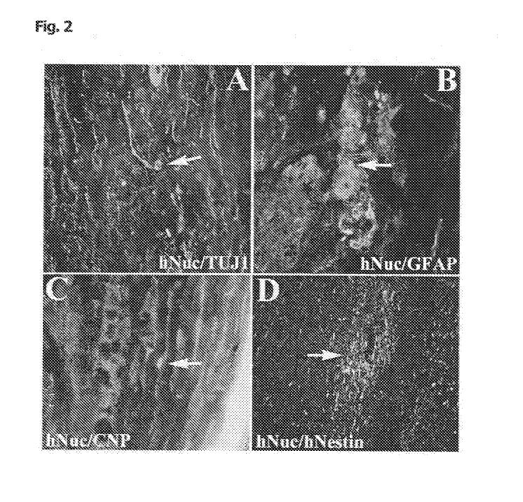

In addition, as shown in FIG. 2, transplanted human neural stem cell immunostained positive to human-specific nuclei antigen (hNuc; Chemicon, Temecula, Calif.) and those shown in red were differentiated into neurons (FIG. 2A), astrocytes (FIG. 2B) and oligodendrocytes (FIG. 2C). Some of the cells were in an undifferentiated state expressing hNestin, a marker for undifferentiated neural stem cell.

Also, BBB (Basso-Beattie-Bresnahan) locomotor rating scale (Basso, et al., J Neurotrauma 1995; 12:1) was used to test the hind limb locomotor skills in experimental group transplanted with human neural stem cell (n=20) and control group injected with H-H buffer solution (n=15), 1 week after the spinal cord injury. The BBB scale was measured each week for 3 months. After 3 months, the average BBB score in human neural cell transplanted group was 11.8±0.4 (average±S.D.) for the left limb and 12.2±0.6 for the right limb. In control group, the average BBB score was 9.0±0.4 for the left limb and 8.6±0.2 for the right limb. The result suggested that the human neural stem cell transplanted group showed a statistically significant increase in locomotor skills (p<0.05).

For an objective analysis of the motor and sensory skills in the stem cell transplanted group and control group, electrophysical tests (Fehlings et al., Electroencephalogr Clin Neurophysiol 1988; 69:65) monitoring motor evoked potential (MEP) and somatosensory evoked potential (SSEP) were performed at 12 weeks post-transplant. According to the SSEP test, the average latency of N1 wave and P1 wave of the control group (3 mice) was 46.7 msec and 68.6 msec and the amplitude was 4.3 μv and 6.2 μv. In the transplanted group (3 mice), the average latency of N1 wave and P1 wave was 36.6 msec and 61.8 msec and the amplitude was 18.9 μv and 33.1 μv. The SSEP result showed shorter latency and higher amplification in the transplanted group compared to the control group, indicating a partial improvement in sensory function in the transplanted group.

According to the MEP test, the average latency of N1 wave and P1 wave of the control group (n=3) was 58.7 msec and 81.5 msec and the amplitude was 1.0 μv and 0.4 μv. In the transplanted group (n=3), the average latency of N1 wave and P1 wave was 49.0 msec and 73.8 msec and the amplitude was 1.5 μv and 2.9 μv. The MEP result showed shorter latency and higher amplification in the transplanted group as compared to the control group, indicating a partial improvement in sensory function in the transplanted group.

When the animals were monitored for 12 months after the human stem cell transplant, the experimental group did not show any abnormal behavior, neurologic manifestation or tumor genesis. Also, there was no tumor, bleeding and abnormal immune reactions detected in the spinal cord injury region and its surrounding area based on the cytological analysis of the animals.

### Example 3

### Human Neural Stem Cell Transplant and Evaluation of the Effect

### 3.1 Transplant Eligible Patients

The eligible patients who received the neural stem cell transplant were quadriplegic with spinal cord injury due to a physical trauma in their cervical spine, were adults aged from 15 to 60, had no previous treatment related to the spine injury, did not have any limb fracture or any associated injury beside the spinal cord injury, did not have any severe surgical and medical disorders that can influence the stem cell transplant and nerve function follow-up, did not have any upper and lower limb joint and muscle atrophy, did not have any progressive/non progressive central and peripheral nervous system disorders, not addicted to drugs or did not have psychiatric disorders. In addition, the patient who had a mechanical spinal cord nerve pressure and required secondary decompression surgery, spinal cord injury in multiple locations, had incompatible factor for transplant according to the physician's decision was eliminated.

### 3.2. Pre-Transplant Testing

Following tests were performed in patients before performing the stem cell transplant procedure. Basic blood and chemical screening tests (CBC, urinalysis, BUN/creatinin, liver function test etc.), physical examination, neurological test including the ASIA (American Spinal Injury Association) 2002 impairment scale assessment and optional elements of sensory and motor neuron (Reference manual for the international standards for neurological classification of spinal cord injury; Braddom R L, Physical medicine & rehabilitation, 3rd edition, Saunders Elesevier, Philadelphia, 2007, pp 1295, 1297-1299; Delisa J A, Phisical medicine & rehabilitation, 4th edition, Lippincott Williams & Wilkins, Philadelphia, 2005, pp 1719-1721), respiration analysis (Kang S W, et al., J Korean Acad Rehab Med 2007; 31:346), spine MRI, evaluation of the pain according to the International association for the Study of Pain (IASP) rating scale (VAS score) (Ohnhaus E E, et al., Pain 1975; 1:379; Wewers M E, et al, Res Nurs Health 1990; 13:227), modified Ashworth scale (Ashworth B, Preliminary trial of carisoprodol in multiple sclerosis, Practitioner 1964; 192:540; Braddom R L. Physical medicine & rehabilitation, 3^{rd} edition. Saunders Elesevier, Philadelphia, 2007, pp 652), electromyography (EMG on both upper and lower limbs) (Liveson J A, Ma D M, Laboratory reference for clinical neurophysiology, F. A. Davis company, Philadelphia, 1992, pp 82-85, 98-100, 133-137, 147-149, 195-200, 204-207, 219-221; Dumitru D, Amato A A, Zwarts M, Electrodiagnostic medicine, 2nd edition, Hanley & Belfus, Philadelphia, 2002, pp 200-204, 211-213) and motor evoked potential (MEP) (Chen R, et al., Clinical Neurophysiology 2008; 119:504; Liveson J A, Ma D M, Laboratory reference for clinical neurophysiology. F. A. Davis company, Philadelphia, 1992, pp 357-362; Dumitru D, Amato A A, Zwarts M, Electrodiagnostic medicine, 2nd edition, Hanley & Belfus, Philadelphia, 2002, pp 419-420), somatosensory evoked potential (SSEP) (Liveson J A, Ma D M, Laboratory reference for clinical neurophysiology. F. A. Davis company, Philadelphia, 1992, pp 278-297, 301-304; Dumitru D, Amato A A, Zwarts M, Electrodiagnostic medicine, 2nd edition, Hanley & Belfus, Philadelphia, 2002, pp 384-395, 400) measured in median nerve, ulnar nerve, tibial nerve, peroneal nerve and pudendal nerve.

When classifying with ASIA (American Spinal Injury Association) 2002 impairment scale, EMG test should be performed to check for no peripheral nerve damage. Complete spinal cord injury (ASIA-A) means loss of sensory and motor functions in sacral spine segment (S4-5) and no reaction from SSEP test. Patients were considered to have an incomplete spinal cord injury (ASIA-B) when they were classified as complete spinal injury by ASIA 2002 scale but an electric potential was detected. Seventeen patient cases with complete motor injury (ASIA-A; n=15, AISA-B; n=2) were enrolled (See Table 1).

### 3.3 Transplant of Human Stem Cells

Anesthesia was administered and the spinal dura was posed by laminectomy technique in patients 2-8 weeks after the spinal cord injury (acute spinal cord injury; 11 cases) and after 8 weeks of the injury (chronic spinal cord injury; 6 cases). With the aid of a surgical microscope, the midline at the epicenter of the spinal cord injury diagnosed with MRI was approached with 23G needle and inserted 5 mm perpendicular to the dorsal part surface. Pre-established human neural stem cell suspension 0.5 ml (1.0×10⁵ μl) was slowly injected over 3 min period. The needle was pulled out after 2 min. In addition, the 5 mm proximal part from the epicenter of the injury and the distal part was each injected with 0.25 ml of human neural cell suspension (1.0×10⁵ μl) for 3 min, similar to the above method.

The immune suppressor cydosporine was administered 3 days pre-transplant (3 mg/kg/day, #2, po) and administered until 2 weeks post-transplant at a same dose. The dosage was reduced to 2 mg/kg/day for the next four weeks, followed by reduction to 1 mg/kg/day for another 2 weeks.

### 3.4 Analysis of the Transplant Follow-Up and Treatment Effect

The following experiments were performed after stem cell transplant as described in example 3.2: blood and chemical screening were performed day 3, week 1, week 2, week 4, week 6, month 2, month 4 and month 6. After the transplant, physical examination, neurological test, ASIA 2002 impairment scale assessment, pain and stiffness were tested every week during week 1 to week 6, then at month 2, month 3, month 6, month 9 and month 12 post-transplant. Spinal cord MRI analysis was performed at week 1, week 8, month 6 and month 12 post-transplant. EMG, SSEP and MEP test was performed at month 2, month 6 and month 12.

The patients received physical and occupational therapy commonly used in spinal cord injury patients, such as standing frame for neurodevelopmental treatment and stationary bicycle for an aerobic exercise. Depending on the level of paralysis, patients were trained for balancing while sitting and ADL training. Patients showing improvement in their motor level had feedback from EMG and received neuromuscular reeducation and pool therapy to stimulate the central pattern generator on their legs.

When monitored over an average of 11 months (12 month and longer; 13 cases, 10 months; 1 case, 7 months; 3 cases, 4 months; 1 case), as shown in FIG. 1, one patient out of 15 ASIA-A patients showed an improvement to the ASIA-B level, and in two patients, their spinal cord injury was improved to ASIA-C level (20% of ASIA-A patients showed improvement in their ASIA scale. The patients who showed improvement were acute spinal cord injury patients. According to the reference, there was a 0-11% change in the ASIA level due to natural regeneration (Bedbrook G M, et al., Paraplegia 1982; 20:321; Frankel H L, et al, Paraplegia 1969; 7:17992; Marino R I, et al., Paraplegia 1995;33:510; Maynard F M, et al., J Neurosurg 1979; 50:611; Stover S L, et al. J Urol 1986; 135:78; Wu L, et al. Arch Phys Med Rehabill 1992; 73:40). In ASIA-B patients, all two patients showed an improvement of their grade to ASID-D level (100% of ASIA-A patients showed improvement in their ASIA scale. The patients who showed improvement were acute spinal cord injury patients. According to the reference, there is 11-14% to 66-89% of various levels of changes in the ASIA level due to natural regeneration (Waters R L, et al., Arch Phys Med Rehabil 1994; 75:306; Crozier K S, et al., Arch Phys Med Rehabil 1991; 72:119; Folman Y, et al., Injury 1989; 20:92; Foo D, et al., Surg Neurol 1981; 15:389; Katoh S, et al., Paraplegia 1995; 33:506) suggesting a clinical improvement of 29% ASIA level change in complete spinal cord injury patients.

However, in the case of 3 patients in ASIA-A patient group (04_Kim, 05_Park and 10_Kwak, Table 1), they showed a severe spinal atrophy according to the surgical opinion, when the human stem cells were injected, the cell suspension leaked out form the spinal cord. Therefore, it is difficult to expect clinical improvements in these patients only by injecting human stem cells. Therefore, these 3 patient cases were excluded. ASIA-A patients showed 25% improvement post-transplant, and there was 36% improvement in complete impairment patients.

In twelve ASIA-A patients who did not show any improvements in ASIA 2002 impairment scale after stem cell transplant (6 cases of acute spinal cord injury, 6 cases of chronic spinal cord injury), all except for 3 cases (chronic spinal cord injury) showed increase in AMS (ASIA motor score) by 5% compared to pre-transplant. Therefore, 75% of ASIA-A patients and over 84% of the complete spinal cord injury patients (14 out of 17 cases) showed improvement in motor function. Even in the ASIA-A group patients (04_Kim, 05_Park and 10_Kwak, Table 1), who showed a severe spinal atrophy according to the surgical opinion, when the human stem cells were injected, the cell suspension leaked out form the spinal cord, except for one patient case (10_Kwak), there was a 5% improvement in motor function after stem cell transplant. There was no stem cell transplant related side effects (bleeding, tumor, pain, stiffness, infection, abnormal immune response, abnormal neurological symptoms, abnormal spinal cord MRI results).

TABLE 1

| | | | | | | | | | | | | |
|---|---|---|---|---|---|---|---|---|---|---|---|---|
| | ASIA | ASIA | Time | | | AMS | | | ASS-L | | | ASS-P |
| Patient | level | level | until | AMS | AMS | improve | ASS-L | ASS-L | improv | ASS-P | ASS-P | improv |
| List | Pre | Post | transplant | Pre | Post | rate | Pre | Post | rate | Pre | post | rate |

No change in ASIA scale (A → A)

| | | | | | | | | | | | | | |
|---|---|---|---|---|---|---|---|---|---|---|---|---|---|
| 1 Shin | A | A | 38 | day | 0 | 6 | 6% | 10 | 12 | 2% | 12 | 12 | 0% |
| 2_Kim | A | A | 46 | day | 0 | 8 | 8% | 11 | 13 | 2% | 11 | 11 | 0% |
| 3_Kwon | A | A | 82 | day | 29 | 38 | 12.7% | 25 | 25 | 0% | 25 | 25 | 0% |
| 4_Kim | A | A | 53 | day | 12 | 24 | 13.6% | 13 | 19 | 6.1% | 13 | 14 | 1% |
| 5_Park | A | A | 141 | day | 13 | 18 | 5.7% | 17 | 18 | 1.1% | 16 | 18 | 2.1% |
| 6_Lim | A | A | 75 | day | 2 | 3 | 1% | 12 | 14 | 2% | 12 | 14 | 2% |
| 7_Lim | A | A | 123 | day | 6 | 6 | 0% | 12 | 12 | 0% | 9 | 12 | 2.9% |
| 8_Kim | A | A | 28 | day | 0 | 5 | 5% | 8 | 15 | 6.7% | 8 | 14 | 5.8% |
| 9_Park | A | A | 59 | day | 26 | 36 | 13.5% | 21 | 31 | 11% | 22 | 24 | 2.2% |
| 10_Kwak | A | A | 7 | Mo | 1 | 1 | 0% | 11 | 12 | 1% | 10 | 12 | 2% |
| 11Whang | A | A | 16 | day | 10 | 26 | 17.8% | 19 | 19 | 0% | 16 | 18 | 2.1% |
| 12_Lim | A | A | 33 | Day | 9 | 18 | 9.9% | 19 | 29 | 10.8% | 16 | 18 | 2.1% |

Change in ASIA scale (A → B, C)

| | | | | | | | | | | | | | |
|---|---|---|---|---|---|---|---|---|---|---|---|---|---|
| 13_Choi | A | C | 21 | day | 15 | 29 | 16.5% | 13 | 16 | 3% | 13 | 22 | 9.1% |
| 14_Lim | A | B | 48 | Day | 18 | 24 | 73% | 22 | 76 | 60% | 19 | 24 | 5.4% |
| 15_Kim | A | C | 18 | day | 9 | 19 | 11% | 10 | 15 | 4.9% | 12 | 26 | 14% |

Change in ASIA scale (B → D)

| | | | | | | | | | | | | | |
|---|---|---|---|---|---|---|---|---|---|---|---|---|---|
| 16_Jun | B | D | 25 | day | 28 | 76 | 66.7% | 68 | 70 | 4.6% | 31 | 46 | 18.5% |
| 17_Kim | B | D | 22 | day | 50 | 72 | 44% | 65 | 94 | 61.7% | 38 | 70 | 43.2% |

### Example 4

Transplantation of Human Neural Stem Cell into a Hypoxic-Ischemic Mouse and Evaluation of the Effect

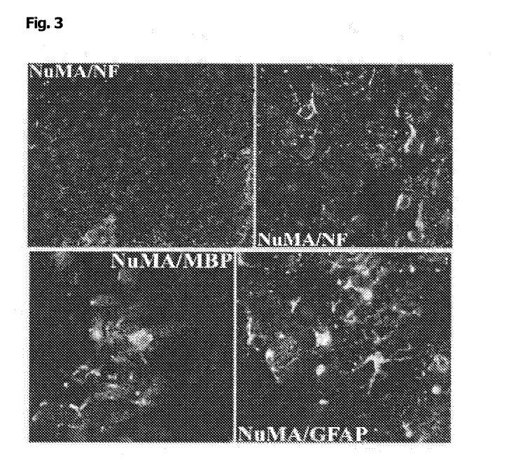

The present invention used the neonatal hypoxic-ischemic mouse model to study the effect of human neural stem cell transplant in regenerating the neonatal hypoxic-ischemic brain injury. Ischemic brain injury was induced on 7 days-old ICR mouse, by exposing the common carotid artery and permanently occluding them. After recovering for 2 hrs, the mice were kept in oxygen and temperature controlled chamber for 1 hr 30 min at 37° C. and an environment of 8% oxygen (Park K I et al., Nat Biotech 2002; 20:1111). One week after the brain injury, the animal model was anesthetized with ketamin (50 mg/kg) and Rompun (10 mg/ml) and the skin on the head was sterilized with 70% alcohol. Using a glass micropipette, 12 μl (1×10⁵ cells/μl) human neural stem cell were injected into the mouse brain infarct area. For control group, 12 μl of H-H buffer was injected into mouse brain infarct area. The surgery area was sterilized with iodine ointment and sutured. The mouse was stabilized on a 37° C. warm pad. To prevent the immune rejection response to human neural stem cell transplanted, cyclosporine (10 mg/kg/day) was administered intraperitonealy one day pre-transplant until the experimental animal died. To analyze the effect of the human neural stem cell on experimental animals, neurological and behavioral test was performed every two weeks starling at 3 weeks after the transplant for up until 11 weeks. At week 11, acquisition of spatial memory and long-term memory was tested. The mouse brain tissue was sampled and analyzed at 12 weeks post-transplant. As shown in FIG. 3, large amounts of human neural stem cells stained immunopositive for human-specific nuclear matrix (hNuMA; Calbiochem, Germany) and shown in red have migrated from the infracted area and engrafted intp distal areas of cerebral cortex, hippocampus, corpus callosum, white matter tract and lateral ventride. Detection of green color by immunopositive staining for neurofilament (NF, Steinberger, USA), Myelin Basic Protein (MBP; DAKO, Carpinteria, Calif.) and glial fibrillary acidic protein (GFAP; DAKO, Carpinteria, Calif.) indicated that the transplanted cells have differentiated into neurons, oligodendrocytes and astrocytes, respectively. Green and red colored dual positive cells are observed in yellow color.

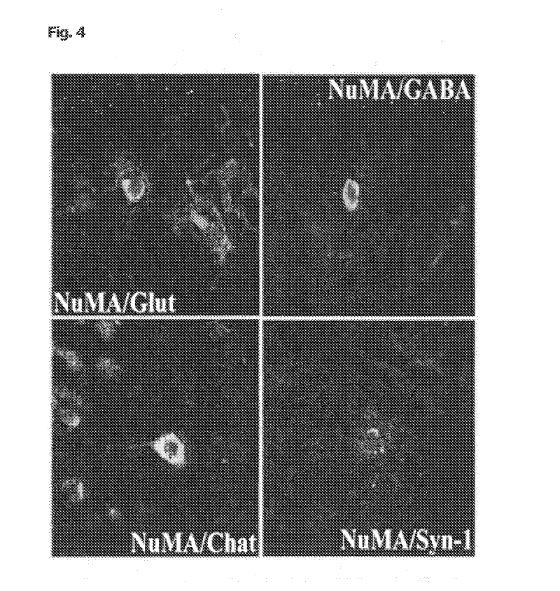

Immunostaining method was used to identify the type of neurotransmitter that was expressed in neuronal cells differentiated from human stem cells transplanted. As shown in FIG. 4, human neural stem cell that were stained immunopositive for hNuMA and detected as red were stained immunopositive for glutamate (Glut; Sigma, Saint Louis, Mo.), γ-Aminobutyric acid (GABA; Sigma, Saint Louis, Mo.) and choline acetyl transferase (Chat; Chemicon, Temecula, Calif.) and was shown in green, indicated that the transplanted cells have differentiated into glutamatergic neuron, GABAnergic neuron and cholinergic neuron, respectively. The red colored hNuMA immunopositive human neural stem cell were detected as green colored Synapsin I (Syn-1; Chemicon, Temicula, Calif.) immunopositive cells, indicating that the human neural stem cell differentiated into neurons have formed synapses. Green and red colored dual positive cells are seen in yellow color.

For the neurological and behavioral test of the animal model, animals were tested for the following six different categories; tail suspension, forelimb flion, torso twisting, right reflection, placing reaction and toe spreading (Brooks and Dunnett, Nat Rev Neurosci 10:519, 2009). Normal movement was graded on a scale as 0 and abnormal movement was graded as 1. As shown in FIG. 5, the neurological test score from experimental animal group transplanted with human neural stem cell (hNSC; n=29) after 3 weeks was 1.14±1.1 (average±SD), after 5 weeks was 0.86±0.99, after 7 weeks was 0.83±0.85, after 9 weeks were 0.76±0.91 and after 11 weeks was 0.62±0.73. In H-H transplanted control group (vehicle; n=33), the neurological test score at 3 weeks was 1.39±1.20, after 5 weeks was 1.45±1.03, after 7 weeks was 1.39±1.00, after 9 weeks was 1.42±1.03 and after 11 weeks was 1.58±1.12.

In summary, a gradual improvement in the disease condition was observed by neurological and behavioral tests in the experimental group transplanted with human neural stem cell. A statistically significant difference was observed at 5 weeks post-transplant when compared with H-H buffer transplanted control group (p<0.05).

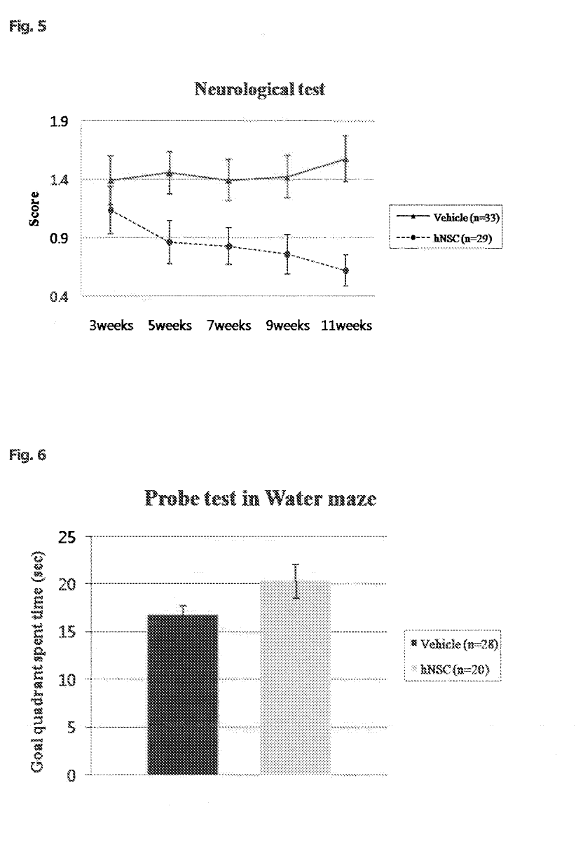

Acquisition of spatial memory and long-term memory (Morris water maze test) was tested 11 weeks after human stem cell transplant (Gerlai, Behav Brain Res 125:269, 2001). The spatial learning session was performed for 6 days in the water tank and on day 7, the time spent in the quadrant was measured (goal quadrant spent time). There was no significant difference between experimental group transplanted with human neural stem cell (hNSC) and H-H buffer transplanted control group during the 6 days of the learning session. However, as shown in FIG. 6, on day 7, the time spent in the quadrant was 20.28±7.83 sec in group transplanted with human neural stem cell (hNSC; n=20) and 16.69±5.24 sec in H-H buffer transplanted control group (vehicle; n=28). Therefore, the result showed that neural stem cell transplanted group had improved spatial memory and therefore had longer latency of time spent in the quadrant. This difference was statistically significant between the two groups (p<0.05).

### Example 5

Transplantation of Human Neural Stem Cell into Refractory Epilepsy Model (Kindling Model) and Evaluation of the Effect

The human neural stem cells were transplanted into the epilepsy model to investigate the effect on inhibiting seizures in epilepsy. In epilepsy model, the one mostly used for temporal lobe epilepsy is the Kindling model and status epilepticus (SE). The kindling model was used for this experiment (Morimoto K, et al., Prog Neurobiol 2004; 73:1). Adult Sprague-Dawley rats (300 mg in body weight) were anesthetized then bipolar electrode was inserted in the dorsal CA3 of right hippocampus. After one week of recovery period, the animal was given electric stimulation (2 msec, 50 Hz, biphasic rectangular, constant current stimulation, 1 sec duration) twice per day. The behavioral changes and change in EEG patterns were monitored using video recording and electroencephalogram (EEG) recording devices. The intensity of the stimulation was determined by the smallest value that indicates the after discharge (AD) in EEG and referred this value as an AD value. AD value was maintained constant during the experiment. The early AD value of stimulation does not induce apparent seizures, but as the stimulation continues, there is a gradually increase in Racine scale ranging from 1 to 6, which represents the severity of the seizures. A kindling model is considered as established when the animal shows a grade 5 in the Racine scale, for 5 consecutive times (Racine R J, Electroenchepalogr Clin Neurophysiol 1972; 32:281, Pinel J P, et al., p Neurol 1978; 58:335, T. Nishimura, et al., Neuroscience 2005; 134:691, McIntyre D C, et al., Epilepsy Res 1993; 14:49, Mirnajafi-Zadeh, et al., Brain Res 2000; 858:48, Vezzani, et al., Neurosci Lett 1988; 87:63). One-week after the generation of the Kindling model, 4 μl (1×10 5 cells/μl) of human stem cells were transplanted. To avoid the immune rejection response against the human neural stem cells transplanted, cyclosporine (10 mg/kg/day) was administered intraperitonealy one day pre-transplant until 8 days after the cell transplant.

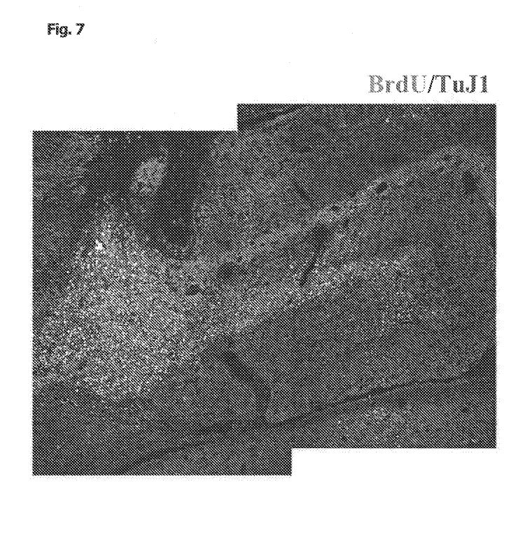

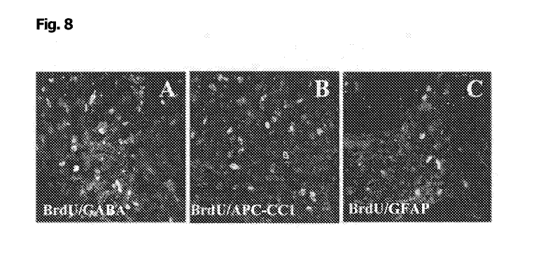

Rat brain tissue was analyzed 2, 4, 8 weeks post-transplant. As shown in FIG. 7, the green BrdU (5-Bromo-2-deoxyuridinel; Roche, USA) immunopositively stained for human neural stem cells migrated and engrafted into the injection site, dorsal hippocampal CA3 region but also into dentate gyrus, which is related in convulsive seizure and fimbriae. The engrafted donor cells expressing red Tuj1 (β-tubulin III; Covance, Berkeley, Calif.) indicated the differentiation into neuronal stem cells. The green BrdU (5-Bromo-2-deoxyuridinel; Roche, USA) immunopositive human neural stem cell expressed red color of immunopositive inhibitory neurotransmitter GAGA (γ-aminobutyrate; Sigma, USA), suggesting that these cells can inhibit the excitatory neurons. Part of the donor cells differentiated into oligodendrocytes (FIG. 8B). In epilepsy models, severe astrogliosis are related to generation and maintenance of convulsive seizure. The transplanted neural stem cell did not differentiate into astrocytes (FIG. 8C) but instead showed a decrease of astrogliosis in host animals transplanted group compared to the control group.

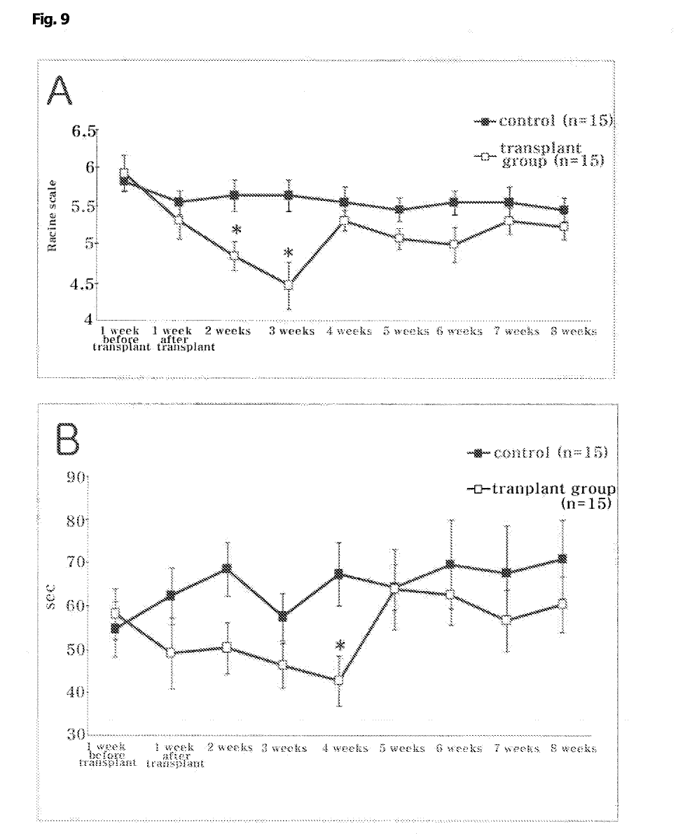

The effect of human neural stem cell on convulsive seizure was studied using refractory epilepsy models transplanted with neural stem cell group (15 rats) and H-H buffer solution injected control group (15 rats). After transplantation, rats were stimulated every week for 8 weeks and the seizures were scored by Racine's scale (FIG. 9A); the duration of status epilepticus was measured by EEG test (FIG. 9B). The level of seizure in stem cell transplanted group gradually decreased and showed a statistical significance when compared to the control group 2, 3 weeks post-transplant (FIG. 9A) (p<0.05). The duration of status epilepticus showed a statistical significant decrease in the transplanted group compared to the control group at 4 weeks post-transplantation (FIG. 9B) (p<0.05).

### Example 6

Transplantation of Human Neural Stem Cell into Alzheimer's Animal Model and Evaluation of the Effect

The human neural stem cell was transplanted into Alzheimer's animal model to analyze the therapeutic efficacy of the neural stem cell on Alzheimer's disease model, the most common form of senile dementia. The Alzheimer's animal model is transgenic mice carrying Swedish mutation (KM595/596NL) in human amyloid precursor protein (APP) 695 isoform gene where the APP is driven by neuron specific enolase (NSE) promoter (Hwang D Y et al., Exp Neurol 2004; 186:20). The transgenic and nontransgenic control mice were generated by crossing with C57BL/6 breeders. The littermates were genotyped 3 weeks after birth and APPsw heterozygote were used as experimental group and nontransgenic littermates were used as controls.

Thirteen-month-old APPsw transgenic mice and normal control mice were anesthetized with xylazine (0.1 mg/10 g of mouse) and ketamine (0.5 mg/10 g of mouse). The head skin was sterilized with 70% of ethanol, incised, and a hole was drilled in the skull bone with 1 mm drill bar on both of the lateral ventride region (0.1 mm anterior, 0.9 mm lateral to Bregma) while fixed on a stereotaxic apparatus. A 10 μl-Hamilton syringe containing 5 μl (1×105 cells/μl or buffer) of human neural stem cell or H-H buffer was inserted 2 mm from the dura mater and slowly transplanted at a speed of 1 μl/min into the lateral ventride space using a micro injection pump. The Hamilton syringe was left for 2 min to avoid leaking then pulled out slowly for the duration of 3 min. The surgery area was sterilized with iodine ointment and sutured. The mouse was stabilized on a 37° C. warm pad. To prevent the immune rejection response against human neural stem cell transplanted, cyclosporine (10 mg/kg/day) was administered intraperitonealy each day, starting one day before the transplant until the experiment!l animal was sacrificed for analysis at week 6. The effect of human stem cell transplant on acquisition of spatial memory and long-term memory was tested 5 weeks after the cell transplant. The mouse brain tissue was sampled and analyzed at week 6.

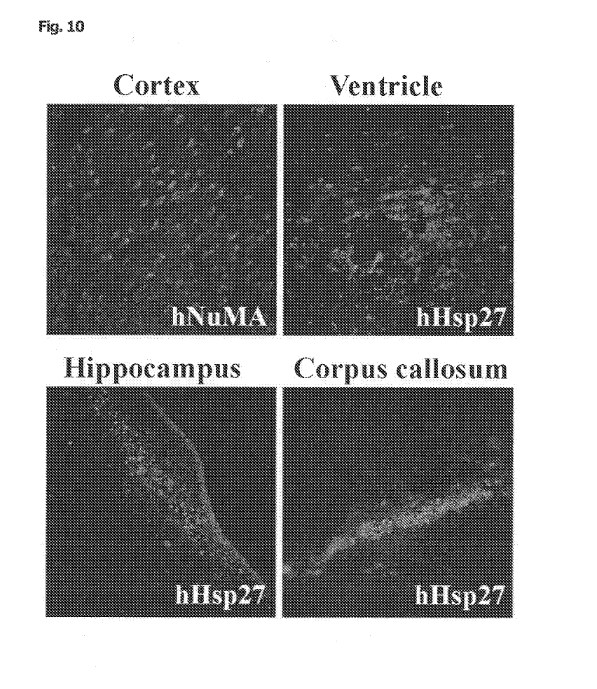

The brain tissue which was analyzed 6 weeks after the transplant (FIG. 10) showed large amounts of human neural stem cells stained immunopositive for human-specific nuclear matrix (hNuMA; Calbiochem, Germany) and hHsp27 (human specific heat shock protein 27; Stressgen, Ann Arbor, Mich.) in red. This immunopositive cells have migrated from the lateral ventricle to distal areas of cerebral cortex, hippocampus and corpus callosum.

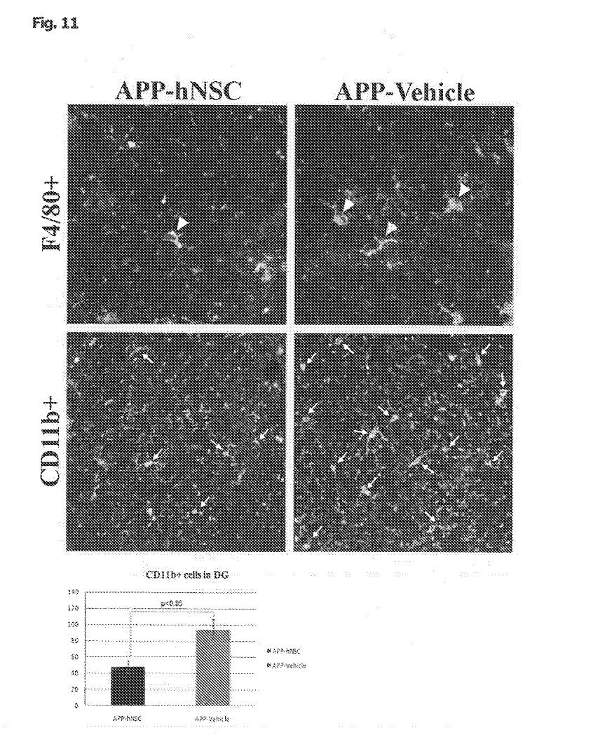

Since inflammatory response has an important pathophysiological function in Alzheimer's disease, the number and the distribution of microglial cells were analyzed in the group where APPsw transgenic mice was transplanted with human neural stem cell (APP-hNSC) and in H-H buffer transplanted control group. Dentate gyms (DG) was immunostained with microglia cell markers, CD11b (AbD Serotec, UK) and F4/80 (AbD Serotec, UK) in transplanted group and control group (FIG. 11). The number of CD11b positive green microglial cells were 48.25±15.08 (average±SD) (n=6), and the number of CD11b positive green microglial cells were 25±24.51 (n=6). The human neural stem cell transplanted group showed statistically significant reduction (p<0.05) of microglia cell markers in dentate gyrus compared to the control group. The transplantation of human neural stem cells reduced the inflammatory response in Alzheimer's disease model.

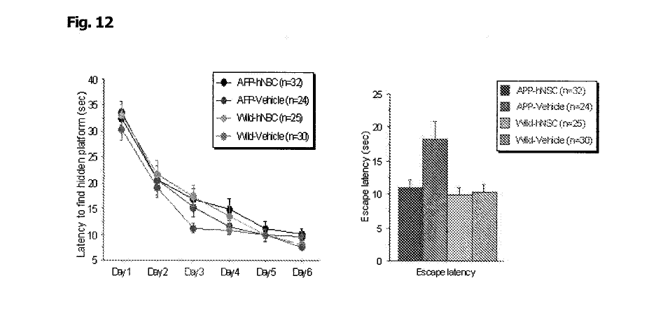

The acquisition of spatial memory and long-term memory was tested in APPsw transgenic mice and control mice, 5 days post-transplant with neural stem cells (hNSC) or the H-H buffer (vehicle). The animals were divided into four experimental groups, group 1; APPsw transgenic mice transplanted with human neural stem cell (APP-hNSC, n=32), group 2; APPsw transgenic mice transplanted with H-H buffer (APP-vehicle, n=24), group 3; wild type mice transplanted with human neural stem cell (Wild-hNSC, n=25) and group 4; wild type mice transplanted with H-H buffer (Wild-vehicle, n=30). The spatial learning session was performed for 6 days before the probe test was performed on day 7. The animals were timed for the escape latency. The four experimental groups did not show significant difference in latency to find hidden platform while trained for six days (FIG. 12A). Escape latency measured on day 7 was 10.98±6.49 sec for APP-hNSC group (average±SD), 18.19±12.96 for APP-vehicle group, 9.83±5.24 for Wild-hNSC group and 10.28±6.26 for Wild-vehicle group. There was a statistically significant improvement of escape latency in APPsw transgenic mice transplanted with human neural stem cells when compare with APPsw transgenic mice transplanted with H-H buffer only (p<0.05). Also, a statistically significant difference was found between APP-vehicle group and Wild-vehicle group (p<0.01), indicating that APPsw transgenic mice had a significantly lower memorizing ability compared to the control mice. However, transplanting neural stem cell into control mice did not show improvement in their memorizing ability.

### INDUSTRIAL APPLICABILITY

As described above, the human neural stem cell of the present invention has active effects on the treatment of patients with nervous system disorders or injuries, particularly on the treatment of patients with spinal cord injury, where currently no treatment is available to reverse the permanent neurological deficits, Parkinson's disease, stroke, amyotrophic lateral sclerosis, motor nerve injury, traumatic peripheral nerve injury, ischemic brain injury, neonatal hypoxic-ischemic brain injury, cerebral palsy, epilepsy, refractory epilepsy, Alzheimer's disease, nervous system disorders, congenital metabolic nervous system disorders, and traumatic brain injury. In addition, the pharmaceutical composition of the present invention comprising the human neural stem cell provides a novel method for treating nervous system disorders.

1. A human neural stem cell assigned with depository number KCTC11370BP.

2-4. (canceled)

5. A method for treating nervous system disorder or injury by administering an effective amount of the human neural stem cell of claim 1 to a subject.

6. The method according to claim 5, where in the nervous system disorder or injury is selected from the group consisting of spinal cord injury, Parkinson's disease, stroke, amyotrophic lateral sclerosis, motor nerve injury, traumatic peripheral nerve injury, ischemic brain injury, neonatal hypoxic-ischemic brain injury, cerebral palsy, epilepsy, refractory epilepsy, Alzheimer's disease, congenital metabolic nervous system disorders, and traumatic brain injury.
